(19) United States
(12) Patent Application Publication
(10) Pub. No.: US 2011/0201569 A1
(43) Pub. Date: Aug. 18, 2011

(54) Methods For Treating Myocardial Disorders

(76) Inventors: Heidi Ehrentraut, Bonn (DE); Hiroshi Shirota, Belmont, MA (US); Georg Baumgarten, Bonn (DE); Pascal Knuefermann, Bonn (DE); Rainer Meyer, Bonn (DE)

(21) Appl. No.: 12/883,707

(22) Filed: Sep. 16, 2010

Related U.S. Application Data
(60) Provisional application No. 61/242,969, filed on Sep. 16, 2009.
(60) Provisional application No. 61/306,312, filed on Feb. 19, 2010.

(51) Int. Cl. A61K 31/7016; A61P 9/00; A61P 9/10; C12N 5/071

(52) U.S. Cl. 514/53; 435/375

(57) ABSTRACT

Provided are methods for treating myocardial disorders comprising administering to a subject an effective amount of a compound of formula (I)

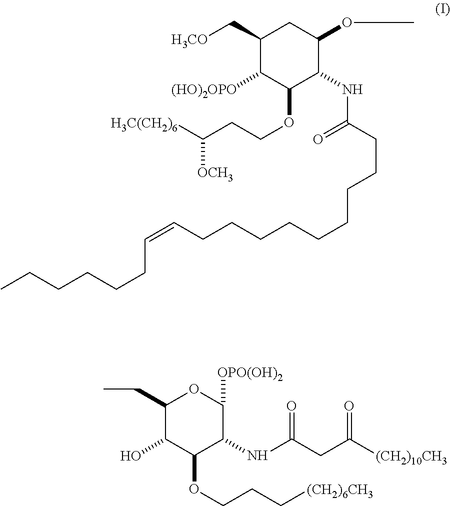

or a pharmaceutically acceptable salt thereof.

### RELATED APPLICATIONS

The present application is related and claims priority to U.S. Provisional Application No. 61/242,969, filed Sep. 16, 2009 and to U.S. Provisional Application No. 61/306,312, filed Feb. 19, 2010. The entire contents of these applications are incorporated herein by this reference.

### BACKGROUND

According to a the National Health and Nutrition Examination Survey (1999-2002) conducted by the Centers for Disease Control and Prevention and National Center for Health Statistics, more than 490,000 Americans die from myocardial infarction each year. Despite much research, the cellular and molecular mechanisms that are involved in myocardial injury in response to ischemia and reperfusion injury are elusive, although the innate immune and inflammatory pathways have been implicated in myocardial ischemia and reperfusion injury and congestive heart failure.

### SUMMARY OF THE INVENTION

The toll like receptor (TLR) family of transmembrane proteins plays a key role in recognizing molecular patterns of pathogens and in triggering the innate immune response. In addition to recognition of molecular patterns of pathogenic organisms, TLRs also bind endogenous ligands. For example, TLRs, and in particular, TLR4, have been recognized as contributors to injury-induced inflammation by binding to endogenous ligands that may be released from injured cells or organs during injury. Endogenous ligands, such as hyaluronic acid, fibronectin, heat shock protein 70 and heparin sulfate, have been found to be released from cells of inflamed tissue and to activate TLR2 and TLR4, initiating or propagating an inflammatory response even in the absence of pathogens. As such, the TLR family has been implicated in myocardial injury.

An effective antagonist of TLR4 is E5564 (also known as eritoran, compound 1287 and SGEA). This drug is described as compound 1 in U.S. Pat. No. 5,681,824, which is incorporated herein by reference. E5564, has the structure of formula (I):

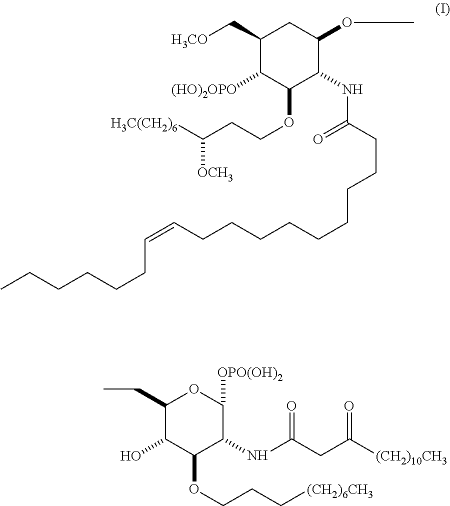

and may be provided as one of a number of sodium salts.

The present teachings relate, at least in part, to the discovery that a compound of formula (A), e.g., a compound of formula (I):

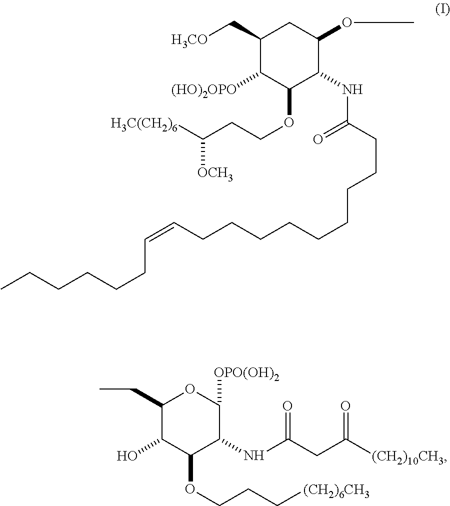

or a pharmaceutically acceptable salt thereof, can be used to treat or prevent myocardial disorders in a subject. The compound of formula (I) has been shown, for example, to influence the development of cardiac hypertrophy, a myocardial disorder, in a mammal model of cardiac hypertrophy.

In some embodiments, provided herein are methods for treating or preventing a myocardial disorder in a subject by administering to the subject an effective amount of a compound of formula (I) or a pharmaceutically acceptable salt thereof.

In some embodiments, the present teachings provide methods for treating a subject at risk of suffering from a myocardial disorder by administering to the subject an effective amount of a compound of formula (I) or a pharmaceutically acceptable salt thereof.

In some embodiments, the myocardial disorder is cardiac hypertrophy or a myocardial infarction. In some embodiments, the cardiac hypertrophy is left ventricle hypertrophy or right ventricle hypertrophy.

In some embodiments, the subject suffers from hypertension, aortic stenosis, hypertrophic cardiomyopathy, emphysema, cystic fibrosis, chronic bronchitis, sleep apnea, chronic pulmonary embolism, heart failure, irregular heart rhythm, angina or cocaine addiction.

In some embodiments, the present teachings provide methods for preventing an increase in myocardial mass in a subject suffering from cardiac hypertrophy by administering to the subject an effective amount of a compound of formula (I) or a pharmaceutically acceptable salt thereof. In some embodiments, the present invention provides methods for decreasing myocardial mass in a subject suffering from cardiac hypertrophy by administering to the subject an effective amount of a compound of formula (I) or a pharmaceutically acceptable salt thereof.

In some embodiments, the present teachings provide methods for decreasing expression or activity of a biomarker of a myocardial disorder in a cell or in a subject suffering from a myocardial disorder by contacting the cell with or administering to the subject an effective amount of a compound of formula (I) or a pharmaceutically acceptable salt thereof. The biomarker of a myocardial disorder can be, for example, atrial natriuretic peptide, brain natriuretic peptide, matrix metalloproteinase-2 and/or matrix metalloproteinase-9

In other embodiments, provided herein are methods for decreasing expression or activity of TLR4 or CD14 in a cell or in a subject suffering from a myocardial disorder by contacting the cell with or administering to the subject an effective amount of a compound of formula (I) or a pharmaceutically acceptable salt thereof.

In other embodiments, the present teachings provide methods for decreasing expression or activity of a cytokine in a cell or in a subject suffering from a myocardial disorder by contacting the cell with or administering to the subject an effective amount of a compound of formula (I) or a pharmaceutically acceptable salt thereof. The cytokine can be, for example, interleukin (IL)-1β, IL-6 and/or IL-10.

In other embodiments, provided herein are methods for treating left ventricle cardiac hypertrophy in a subject by administering to the subject an effective amount of a compound of formula (I) or a pharmaceutically acceptable salt thereof.

### BRIEF DESCRIPTION OF THE DRAWINGS

FIGS. 1(A), (B) and (C) include graphic comparisons of left ventricular weight, left ventricular weight/body weight (LVW/BW), and left ventricular weight/tibia length (LVW/TL) (*p<0.05; M±SEM; n=6-9).

FIGS. 2(A) and (B) include graphic comparisons of the mRNA expression of atrial natriuretic peptide (ANP) and brain natriuretic peptide (BNP), respectively, found in cardiac tissue of the test subjects (*p<0.05; M±SEM; n=5-7).

FIGS. 3(A), (B) and (C) include graphic comparisons of (A) the mRNA expression of matrix metalloproteinase-2, matrix metalloproteinase-9 and total matrix metalloproteinase (MMP-2+MMP-9) in cardiac tissue of the test subjects (*p<0.05; M±SEM; n=5-7 (MMP-9), n=4-5 (MMP-2, total MMP)).

FIGS. 4(A) and (B) include graphic comparisons of (A) the mRNA-expression of and TIMP-1 and TIMP-4 in cardiac tissue of the test subjects and (B) MMP-2 and -9 activity in gelatine zymography (*p<0.05; M±SEM; n=5-7).

FIGS. 5(A), (B) and (C) include graphic comparisons of the mRNA expression of cytokines IL-1β, IL-6 and IL-10, respectively, in cardiac tissue of the test subjects (M±SEM; n=5-7).

FIGS. 6(A) and (B) include graphic comparisons of the protein expression of cytokines IL-1β and IL-6, respectively, in cardiac tissue of the test subjects (M±SEM; n=5-7).

FIGS. 7(A) and (B) include graphic comparisons of mRNA expression of TLR4 and its co-receptor CD14, respectively, in cardiac tissue of the test subjects (*p<0.05, M±SEM; n=5-7).

FIG. 8 includes a graphic comparison of left ventricular weight among the test subjects (LVW; *:p<0.05, n=6/group).

FIGS. 9(A) and (B) include graphic comparisons of the mRNA expression of ANP and BNP, respectively, found in cardiac tissue of the test subjects (*:p<0.05, n=6/group).

FIGS. 10(A) and (B) include graphic comparisons of the mRNA expression of matrix metalloproteinase-2 and matrix metalloproteinase-9, respectively, in cardiac tissue of the test subjects (n=6/group).

FIGS. 11(A) and (B) include graphic comparisons of mRNA expression of TLR4 and its co-receptor CD14, respectively, in cardiac tissue of the test subjects (*:p<0.05, n=6/group).

FIGS. 12(A), (B) and (C) include graphic comparisons of mRNA expression of cytokines IL-1β, IL-6 and IL-10, respectively, in cardiac tissue of the test subjects (n=6/group).

FIGS. 13(A) and (B) include a graphic comparison of the protein expression of cytokines IL-1β and IL-6, respectively, in cardiac tissue of the test subjects (n=6/group).

FIGS. 14(A) and (B) include a graphic comparison of mRNA expression of tissue inhibitors of matrix metalloproteinase 1 (TIMP-1) and tissue inhibitors of matrix metalloproteinase 4 (TIMP-4), respectively, in cardiac tissue of the test subjects (n=6/group).

### DETAILED DESCRIPTION OF THE INVENTION

The present teachings are based, at least in part, on the discovery that the compounds described herein are able to effectively treat myocardial disorders, such as cardiac hypertrophy. Presently, treatment for cardiac hypertrophy depends on the condition of the heart and the severity of symptoms, and is intended to decrease stress on the heart and relieve symptoms. Such treatment includes, for example, surgery (e.g., myectomy or septal ablation), medical devices (e.g., automatic implantable defibrillator or pacemaker) or medications to relax the heart (e.g., beta-blockers, calcium channel blockers and anti-arrhythmia drugs). In some embodiments, the compounds described herein are able to affirmatively treat myocardial disorders such as cardiac hypertrophy without the use of surgery or medical devices.

### Definitions

In order to more clearly and concisely describe the subject matter of the claims, the following definitions are intended to provide guidance as to the meaning of terms used herein.

As used herein, the articles “a” and “an” mean “one or more” or “at least one,” unless otherwise indicated. That is, reference to any element of the present invention by the indefinite article “a” or “an” does not exclude the possibility that more than one of the element is present.

Certain values and ranges are recited in connection with various embodiments of the present invention, e.g., amount of a compound of formula (I) present in a composition. It is to be understood that all values and ranges which fall between the values and ranges listed are intended to be encompassed by the present invention unless explicitly stated otherwise.

The term “about” as used herein in association with parameters, ranges and amounts, means that the parameter or amount is within ±1% of the stated parameter or amount.

As used herein, the term “subject” refers to animals such as mammals, including, but not limited to, humans, primates, cows, sheep, goats, horses, pigs, dogs, cats, rabbits, guinea pigs, rats, mice or other bovine, ovine, equine, canine, feline, rodent or murine species. In some embodiments, the subject is a human. In some embodiments, the subject is a human athlete.

As used herein, “alkyl” groups include saturated hydrocarbons having one or more carbon atoms, including straight-chain alkyl groups (e.g., methyl, ethyl, propyl, butyl, pentyl, hexyl, methylene, ethylene, propylene, butylene, pentylene, hexylene, etc.), cyclic alkyl groups (or “cycloalkyl” or “alicyclic” or “carbocyclic” groups) (e.g., cyclopropyl, cyclopentyl, cyclohexyl, etc.), branched-chain alkyl groups (isopropyl, tert-butyl, sec-butyl, isobutyl, etc.), and alkyl-substituted alkyl groups (e.g., alkyl-substituted cycloalkyl groups and cycloalkyl-substituted alkyl groups). The term “C1 to C6” as in “C1 to C6 alkyl” means alkyl groups containing 1 to 6 carbon atoms.

“Treatment”, “treat”, or “treating” as used herein, are defined as the application or administration of a therapeutic agent (e.g., a compound of formula (A) or a compound of formula (I)) to a subject, or to an isolated tissue or cell line from a subject. The subject generally has a disease or disorder, a symptom of disease or disorder or a predisposition toward a disease or disorder (e.g., a myocardial disorder). The purpose of treatment is generally to cure, heal, alleviate, relieve, remedy, ameliorate, or improve such disease, disorder, symptoms or predisposition. “Treated”, as used herein, refers to the disease or disorder being cured, healed, alleviated, relieved, remedied, ameliorated, or improved. For example, methods of treatment of the instant teachings provide for administration of a compound of formula (A) or formula (I), as described herein, such that a myocardial disorder (e.g., cardiac hypertrophy or myocardial infarction) is slowed or stopped. Methods of treatment of the instant teachings further include the administration of the compound of compound of formula (A) or the compound of formula (I) such that the myocardial disorder is cured.

As used herein, the term “cell” refers any animal cells. In some embodiments, cells include, but are not limited to blood cells, the cells that make up the blood vessels and arteries, cells that line the blood vessels and arteries and the muscle cells of the heart tissue (e.g., cardiac myocytes).

As used herein, the terms “prevent,” prevention” or “preventing” refer to inhibiting a biological response completely or partially, as well as inhibiting an increase in a biological response. For instance, prevention of cardiac hypertrophy refers to partially or completely inhibiting cardiac muscle enlargement, as well as inhibiting the progression of cardiac muscle enlargement. Thus the term prevention embraces the use of the compounds for inhibiting an response before it begins or treating a subject in which a response has already begun in order to slow or stop the progression. A person of ordinary skill in the art would recognize that the term “prevent” is not an absolute term, but is rather understood to refer to the prophylactic administration of a drug to substantially diminish the likelihood or seriousness of a condition. That is, the term “prevent” does not require that the disorder be completely thwarted. Rather, preventing refers to the ability of the skilled artisan to identify a population that is susceptible to a myocardial disorder as described herein, such that administration of a compound of formula (A), a compound of formula (I), or a pharmaceutically acceptable salt thereof, may occur prior to the onset of the symptoms of the myocardial disorder.

Unless otherwise defined, all technical and scientific terms used herein have the same meaning as commonly understood by one of ordinary skill in the art. In case of conflict, the present application, including definitions will control.

### Myocardial Disorders

In some embodiments, the present teachings provide methods for treating or preventing a myocardial disorder in a subject in need thereof by administering to the subject an effective amount of a compound of formula (A), e.g., a compound of formula (I), or a pharmaceutically acceptable salt thereof. In some embodiments, the present teachings provide methods for treating a subject at risk of suffering from a myocardial disorder by administering to the subject an effective amount of a compound of formula (A), e.g., a compound of formula (I), or a pharmaceutically acceptable salt thereof.

As used herein, the term “myocardial disorder” refers to a disorder associated with the myocardial tissue of the heart (e.g., the heart muscle), for example, due to deformity of, injury to or malfunction of the myocardial tissue. Examples of myocardial disorders include, but are not limited to, cardiomyopathy and myocardial infarctions. In some embodiments, the myocardial disorder is cardiac hypertrophy or a myocardial infarction.

In some embodiments, the present teachings provide methods for treating or preventing cardiomyopathy in a subject. As used herein, the term “cardiomyopathy” refers to a weakening of the heart muscle or a change in heart muscle structure. Examples of cardiomyopathies include, for example, alcoholic cardiomyopathy, dilated cardiomyopathy, cardiomyopathy associated with celiac disease, cardiac hypertrophy, cardiomyopathy associated with nutritional deficiencies, cardiomyopathy associated with kidney disease, cardiomyopathy associated with systemic lupus erythematosus, peripartum cardiomyopathy, tachycardia mediated cardiomyopathy, idiopathic cardiomyopathy, hypertensive cardiomyopathy, infectious cardiomyopathy, toxic cardiomyopathy and restrictive cardiomyopathy.

The term “alcoholic cardiomyopathy,” as used herein, refers to a condition in which the heart muscle weakens or changes structure due to the long term use of alcohol. The term “dilated cardiomyopathy,” as used herein, refers to a condition in which the chambers of the heart dilate, leading to progressive contractile dysfunction and a weakening of the myocardial tissue due to stretching of the tissue. As used herein, the term “cardiomyopathy associated with nutritional deficiencies” refers to a condition in which the heart muscle weakens or changes structure due to the absence of certain vitamins and minerals in the diet, for example, selenium, thiamine or L-carnitine. The term “peripartum cardiomyopathy,” as used herein, refers to a condition in which the heart muscle weakens or changes structure during the last month of pregnancy or during the first five months after giving birth.

As used herein, the term “tachycardia mediated cardiomyopathy” refers to a condition in which the heart muscle weakens or changes structure due to an abnormally fast heartbeat. As used herein, the term “idiopathic cardiomyopathy” refers to a condition in which the heart muscle weakens or changes structure due to unknown causes. As used herein, the term “hypertensive cardiomyopathy” refers to a condition in which the heart muscle weakens or changes structure due to hypertension (e.g., high blood pressure). As used herein, the term “infectious cardiomyopathy” refers to a condition in which the heart muscle weakens or changes structure due to infections, for example, HIV/AIDS, Lyme disease, Chagas disease or viral myocarditis. As used herein, the term “toxic cardiomyopathy” refers to a condition in which the heart muscle weakens or changes structure due to cocaine use or use of chemotherapy drugs. As used herein, the term “restrictive cardiomyopathy” refers to a condition in which the heart chambers are unable to properly fill with blood due to stiffness in the myocardial tissue.

In some embodiments, the present teachings provide methods for treating or preventing cardiac hypertrophy in a subject. The term “cardiac hypertrophy” refers to a condition in which the heart muscle thickens due to an increase in the size of the myocardial cells. In some embodiments, the cardiac hypertrophy is left ventricle cardiac hypertrophy. The term “left ventricle cardiac hypertrophy” refers a disorder in which the myocardial tissue of the left ventricle of the heart thickens. Without being bound by theory, causes of left ventricle cardiac hypertrophy include, for example, hypertension (e.g., high blood pressure), stenosis of the aortic valve (e.g., the inability of the heart valve to fully open), and hypertrophic cardiomyopathy (e.g., a disorder in which the myocardial tissue thickens for no obvious cause). In other embodiments, the cardiac hypertrophy is right ventricle cardiac hypertrophy. The term “right ventricle cardiac hypertrophy” refers to a disorder in which the myocardial tissue of the right ventricle thickens. Without being bound by theory, causes of right ventricle hypertrophy include, for example, diseases that damage the lungs, such as emphysema and cystic fibrosis; conditions that decrease oxygen levels in the body, such as chronic bronchitis and sleep apnea; stenosis of the pulmonic heart valve, chronic pulmonary embolism and primary pulmonary hypertension.

In some embodiments, the present teachings are directed to the treatment of cardiac hypertrophy associated with hypertension. Murine transverse aortic constriction (TAC) is an experimental model that mimics hypertensive remodeling in human hypertension. Reducing the aortic diameter to 30% with TAC surgery exposes the left ventricle to pressure overload and subsequently induces a compensatory left ventricular hypertrophy. The inventors have demonstrated that treatment with a compound of formula (I), or a pharmaceutically acceptable salt thereof, reduces cardiac hypertrophy after aortic banding in vivo.

In some embodiments, the myocardial disorder is a myocardial infarction. The terms “myocardial infarction” (MI), “acute myocardial infarction” (AMI) and “heart attack,” as used herein, refer to the interruption of blood supply to part of the heart, causing necrosis and death of the myocardial tissue. The term “acute myocardial infarction” includes both transmural MI and non-transmural MI. The term “transmural MI” refers to the ischemic necrosis of the full thickness of the affected muscle segments or segments, extending from the endocardium through the myocardium to the epicardium. The term “non-transmural MI” refers to the area of ischemic necrosis that is limited to the endocardium or endocardium and myocardium.

In some embodiments, the myocardial disorder is not cardiac failure (e.g., a condition in which the heart can no longer pump enough blood to the rest of the body), a cardiac surgical procedure (e.g., coronary bypass, valve replacement or coronary artery graft), a cardiovascular disease (e.g., atherosclerosis or arteriosclerosis), cardiopulmonary bypass associated morbidity or mortality of hypertension.

In some embodiments, the subject is at risk of suffering from a myocardial disorder. As used herein, the term “at risk of suffering from a myocardial disorder” refers to a subject that may have a predisposition for a myocardial disorder.

In some embodiments, the subject suffers from hypertension, aortic stenosis, hypertrophic cardiomyopathy, emphysema, cystic fibrosis, chronic bronchitis, sleep apnea, chronic pulmonary embolism, heart failure, irregular heart rhythm, angina, alcoholism or cocaine addiction.

Treatment according to the present teachings can include administration of compounds described herein in an effective amount. As used herein, the term “effective amount” refers to the amount of the compound of formula (A) (e.g., the compound of formula (I)) necessary to achieve a desired effect. The term “desired effect” refers generally to any result that is anticipated by the skilled artisan when the compound described herein is administered to a subject or a cell. In some embodiments, the desired effect is the treatment or prevention of a myocardial disorder in a subject. In other embodiments, the desired effect is the treatment of a myocardial disorder in a subject at risk of suffering from a myocardial disorder. In still other embodiments, the desired effect is the complete elimination of the myocardial disorder. In other embodiments, the desired effect is the prevention of the increase in myocardial mass in a subject suffering from cardiac hypertrophy. In yet other embodiments, the desired effect is the decrease in expression or activity of a biomarker of a myocardial disorder (e.g., atrial natriuretic peptide, brain natriuretic peptide, matrix metalloproteinase-2 or matrix metalloproteinase-9) in a subject or a cell. In still other embodiments, the desired effect is the decrease in expression or activity of TLR4 or CD14 in a subject or a cell. In some embodiments, the desired effect is the decrease in expression or activity of a cytokine (e.g., IL-1β, IL-6 or IL-10) in a subject or a cell. In still other embodiments, the desired effect is the treatment of left ventricle cardiac hypertrophy in a subject. The exact amount of the compound of formula (A) or formula (I) required will vary from subject to subject, depending on the species, age, and general condition of the subject, the severity of the diseases, its mode of administration, and the like.

In some embodiments, the effective amount is an effective periodic dose. As used herein, the term “effective periodic dose” refers to the amount effective to treat or prevent a myocardial disorder in a subject, which dose is administered in periodic intervals over time. In some other embodiments, the effective periodic dose is the amount effective to treat a myocardial disorder in a subject at risk of suffering from a myocardial disorder. In still other embodiments, the effective periodic dose is the amount effective to completely eliminate the myocardial disorder. In some other embodiments, the effective periodic dose is the amount effective to prevent an increase in myocardial mass in a subject suffering from cardiac hypertrophy. In yet other embodiments, the effective periodic dose is the amount effective to decrease expression or activity of a biomarker of a myocardial disorder (e.g., atrial natriuretic peptide, brain natriuretic peptide, matrix metalloproteinase-2 or matrix metalloproteinase-9) in a subject or a cell. In still other embodiments, the effective periodic dose is the amount effective to decrease expression or activity of TLR4 or CD14 in a subject or a cell. In some embodiments, the effective periodic dose is the amount effective to decrease expression or activity of a cytokine (e.g., IL-1β, IL-6 or IL-10) in a subject or a cell. In yet other embodiments, the effective periodic dose is the amount effective to treat left ventricle cardiac hypertrophy in a subject.

In other embodiments, the effective periodic dose is a once daily dose, a twice daily dose, a thrice daily dose, a twice weekly dose, a weekly dose, a bi-weekly dose, or a monthly dose. In some embodiments, the effective periodic dose is administered for about 1 day, for about 3 days, for about a week, for about 1 month, for about 3 months, for about 6 months, for about 9 months, for about 1 year or for greater than about 1 year. In other embodiments, the effective period dose is the highest tolerable dose that could be administered safely. The exact amount required will vary from subject to subject, depending on the species, age, and general condition of the subject, the severity of the diseases, its mode of administration, and the like.

In some embodiments, the present teachings provide methods for preventing an increase in the myocardial mass in a subject by administering to the subject an effective amount of a compound described herein. As used herein, the term “preventing an increase in myocardial mass” refers to the ability of the compound of formula (A) or formula (I) to inhibit the physiological processes that lead to an increase in mass of the myocardial tissue. In some embodiments, the mass of the myocardial tissue in a subject is prevented from increasing mass by about 5%, by about 10%, by about 15% or by about 20% upon treatment with a compound of formula (I) compared to a subject that has not been treated with a compound of formula (I).

In some embodiments, provided herein are methods for decreasing the myocardial mass in a subject by administering to the subject an effective amount of a compound described herein. In some embodiments, the mass of the myocardial tissue in a subject is decreased by about 5%, by about 10%, by about 15% or by about 20% upon treatment with a compound described herein.

### Compounds

In some embodiments, compounds of formula (A) and pharmaceutically acceptable salts thereof are used in connection with the present teachings. Compounds of formula (A) have the following structure:

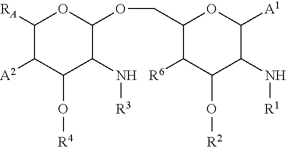

and pharmaceutically acceptable salts thereof
wherein R¹ is selected from:

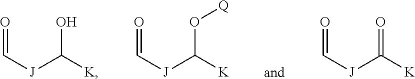

where J is straight or branched C1 to C3 alkyl; K is straight or branched C8 to C15 alkyl; and Q is straight or branched C1 to C3 alkyl;
R² is straight or branched C8 to C12 alkyl;
R³ is selected from:

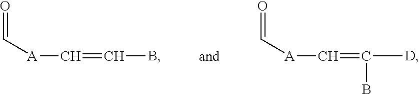

where A is straight or branched C7 to C12 alkyl; and each B and D, independently, is straight or branched C4 to C9 alkyl;
R⁴ is selected from:
   straight or branched C8 to C12 alkyl, and

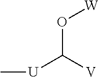

where U is straight or branched C2 to C4 alkyl; V is straight or branched C5 to C9 alkyl; and W is hydrogen or —CH₃;
R_{A} is R⁵—O—CH₂—, where R⁵ is hydrogen or straight or branched C1 to C5 alkyl;
R⁶ is hydroxy; and
A¹ and A² are each independently

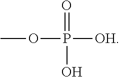

In some embodiments, R¹ is:

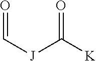

where J is straight or branched C1 to C3 alkyl; K is straight or branched C8 to C15 alkyl; and Q is straight or branched C1 to C3 alkyl. In some embodiments, J is a C1 alkyl, e.g., —CH₂—. In some embodiments, K is a C10 to C12 alkyl, e.g., a C11 alkyl.

In some embodiments, R² is straight or branched C8 to C12 alkyl, e.g., a C9 to C11 alkyl, e.g., a C10 alkyl.

In some embodiments, R³ is:

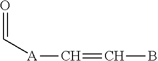

where A is straight or branched C7 to C12 alkyl; and B is straight or branched C4 to C9 alkyl. In some embodiments, A is a C8 to C11 alkyl, e.g., a C9 alkyl. In some embodiments, B is a C5 to C8 alkyl, e.g., a C6 alkyl.

In some embodiments, R⁴ is:

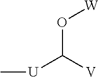

where U is straight or branched C2 to C4 alkyl; V is straight or branched C5 to C9 alkyl; and W is hydrogen or —CH₃. In some embodiments, U is C2 alkyl, e.g., —CH₂CH₂—. In some embodiments, V is a C6 to C8 alkyl, e.g., a C7 alkyl. In some embodiments, W is a —CH₃.

In some embodiments, R_{A} is R⁵—O—CH₂—, where R⁵ is straight or branched C1 to C5 alkyl. In some embodiments, R5 is a C1 alkyl, e.g., —CH₃.

In some embodiments, compounds of formula (A) have the following structure:

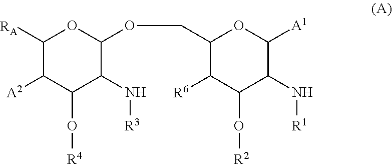

or pharmaceutically acceptable salts thereof
where R¹ is:

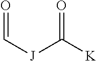

where J is straight or branched C1 to C3 alkyl; K is straight or branched C8 to C15 alkyl; and Q is straight or branched C1 to C3 alkyl;
R² is straight or branched C8 to C12 alkyl;
R³ is:

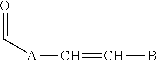

where A is straight or branched C7 to C12 alkyl; and B is straight or branched C4 to C9 alkyl;
R⁴ is:

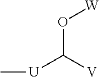

where U is straight or branched C2 to C4 alkyl; V is straight or branched C5 to C9 alkyl; and W is hydrogen or —CH₃;
R_{A} is R⁵—O—CH₂—, where R⁵ is straight or branched C1 to C5 alkyl;
R⁶ is hydroxy; and
A¹ and A² are each independently

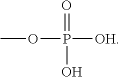

In some embodiments, compounds of formula (I) and pharmaceutically acceptable salts thereof are used in connection with the present teachings. The term “compound of formula (I)” refers to a compound having the structure:

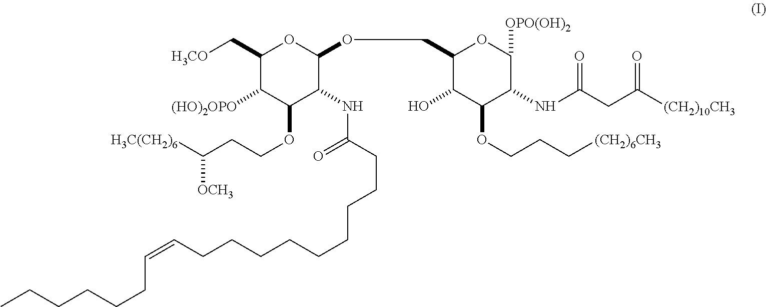

or a pharmaceutically acceptable salt thereof. The compound of formula (I) may also be known as E5564, 1287, eritoran, SGEA or (α-D-Glucopyranose, 3-O-decyl-2-deoxy-6-O-[2-deoxy-3-O-[(3R)-3-methoxydecyl)-6-O-methyl-2-[[(−11Z)-1-oxo-11-octadecenyl)amino]-4-O-phosphono-β-D-glucopyranosyl]-2-[(1,3-dioxotetradecyl)amino]-1-(dihydrogen phosphate). The compound of formula (I) is described as compound 1 in U.S. Pat. No. 5,681,824, which is incorporated herein by reference.

The compound of formula (A), e.g., the compound of formula (I), may be prepared in the form of a micelle, as described in U.S. Pat. No. 6,906,042, which is incorporated herein by reference in its entirety for the description of such micelles and methods for preparing same. In some embodiments, the compounds described herein are administered in a formulation which includes micelles having a mean hydrodynamic diameter of between about 7 nm and about 15 nm In some embodiments, the compounds described herein are administered in a formulation which includes micelles having a mean hydrodynamic diameter of between about 7 nm and about 14 nm, between about 7 nm and about 13 nm, between about 7 nm and about 12 nm, or between about 7 nm and about 11 nm. In some embodiments, the compounds described herein are administered in a formulation which includes micelles having a mean hydrodynamic diameter of between about 7 nm and about 10 nm In some embodiments, the compounds described herein are administered in a formulation which includes micelles having a mean hydrodynamic diameter of between about 7 nm and about 9 nm.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al., describe pharmaceutically acceptable salts in detail in J. Pharmaceutical Sciences, 66: 1-19 (1977), which is incorporated herein by reference. The salts can be prepared in situ during the final isolation and purification of the compounds taught herein, or separately by reacting a free base or free acid function with a suitable reagent, as described generally below. For example, a free base function can be reacted with a suitable acid. Furthermore, where the compounds taught herein carry an acidic moiety, suitable pharmaceutically acceptable salts thereof may, include metal salts such as alkali metal salts, e.g., sodium or potassium salts; and alkaline earth metal salts, e.g., calcium or magnesium salts. Sodium salts of compounds within the scope of Compound I are described, for example, in U.S. patent application Ser. No. 12/516,082 and U.S. Pat. Application Publication No. 2008/0227991. Examples of pharmaceutically acceptable, nontoxic acid addition salts are salts formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. In some embodiments, the compound of formula (I) is a sodium salt, e.g., a tetrasodium salt.

It will be appreciated that according to the methods taught herein, the compound of formula (A) or the compound of formula (I) may be administered using any amount and any route of administration effective for treating or preventing a myocardial disorder in a subject, preventing the increase in myocardial mass in a subject, decreasing the expression or activity of a biomarker of a myocardial disorder (e.g., atrial natriuretic peptide, brain natriuretic peptide, matrix metalloproteinase-2 or matrix metalloproteinase-9) in a subject or a cell, decreasing the expression or activity of TLR4 or CD14 in a subject or a cell, decreasing the expression or activity of a cytokine (e.g., IL-1β, IL-6 or IL-10) or treating left ventricle cardiac hypertrophy in a subject. It will be understood, however, that the administration of the compound of formula (A) or formula (I) will be decided by the attending physician within the scope of sound medical judgment. The specific therapeutically effective dose level for any particular subject or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics,” Tenth Edition, A. Gilman, J. Hardman and L. Limbird, eds., McGraw-Hill Press, 155-173, 2001, which is incorporated herein by reference in its entirety).

In some embodiments, the compounds described herein are administered systemically. As used herein, “systemic administration” refers to any means by which the compounds described herein can be made systemically available. In some embodiments, systemic administration encompasses intravenous administration, intraperitoneal administration, intramuscular administration, intracoronary administration, intraarterial administration (e.g., into a carotid artery), intradermal administration, subcutaneous administration, transdermal delivery, intratracheal administration, subcutaneous administration, intraarticular administration, intraventricular administration, inhalation (e.g., aerosol), intracerebral, nasal, naval, oral, intraocular, pulmonary administration, impregnation of a catheter, by suppository and direct injection into a tissue, or systemically absorbed topical or mucosal administration. Mucosal administration includes administration to the respiratory tissue, e.g., by inhalation, nasal drops, ocular drop, etc.; anal or vaginal routes of administration, e.g., by suppositories; and the like. In some embodiments, the compounds described herein are administered intravenously. In other embodiments, the compounds described herein are administered orally. In some embodiments, the compounds described herein may be administered intravenously one to five times a week. In some other embodiments, the compounds described herein may be administered orally one or more times a day (e.g., once a day, twice a day or three times a day).

### Altering Gene/Protein Expression

In some embodiments, the present teachings provide methods for decreasing the expression, e.g., the level or amount, and/or activity of a gene and/or protein of interest, e.g., a biomarker, in a subject by administering to the subject an effective amount of a compound of formula (A) or a compound of formula (I). In some embodiments, the present teachings provide methods for decreasing the expression, e.g., the level or amount, and/or activity of a biomarker in a cell by contacting the cell with an effective amount of a compound of formula (A) or a compound of formula (I). As used herein, the term “biomarker” is intended to encompass a substance that is used as an indicator of a biologic state, e.g., of a myocardial disorder, and includes genes (and nucleotide sequences of such genes), mRNAs (and nucleotide sequences of such mRNAs) and proteins (and amino acid sequences of such proteins). In some embodiments, a biomarker is a biomarker of a myocardial disorder and includes, but is not limited to, atrial natriuretic peptide (ANP), brain natriuretic peptide (BNP), matrix metalloproteinase-2 (MMP-2), matrix metalloproteinase-9 (MMP-9), tissue inhibitor of metalloproteinase-1 (TIMP-1) and tissue inhibitor of metalloproteinase-4 (TIMP-4).

Without wishing to be bound by any particular theory, it is believed that cardiac hypertrophy, e.g., TAC-induced hypertrophy, causes the increase of natriuretic peptides ANP and BNP. These natriuretic peptides are secreted in response to muscle stretching and act locally at the sites of their synthesis. Thus, they are useful clinical markers for hypertrophy and cardiac dysfunction, correlating with the severity of symptoms and prognosis (see, e.g., Gerber et al., Circulation 2003, 107: 1884-1890 and Ritchie et al., Curr Mol Med 2009, 9: 814-825). Between the two, BNP is typically a more sensitive marker for cardiac dysfunction.

Without wishing to be bound by any particular theory, it is also believed that activation of matrix metalloproteinases (MMPs) and imbalances of MMPs and tissue inhibitors of metalloproteinase (TIMPs) are associated with changes in the extracellular matrix composition, causing cardiac remodeling, fibrosis, and finally dysfunction (see, e.g., Heymans et al., Am J Pathol 2005, 166: 15-25 and Lee et al., Trends Cardiovasc Med 2001, 11: 202-205). Previous publications reported that inhibition of MMPs in early stages of hypertrophic development prevented left ventricular hypertrophy. Moreover, increased MMP-9 activity promoted the transition to decompensated cardiac heart failure (see, e.g., Graham et al., Am J Physiol Heart Circ Physiol 2007, 292: H1364-H1372), and MMP-9 deletion protected mice from cardiac fibrosis and dysfunction after aortic banding. Thus, they are also useful clinical markers for hypertrophy and cardiac dysfunction.

As used herein, the term “decreasing the expression”, e.g., of a biomarker of a myocardial disorder, refers to the reduction in the level or amount of biomarker that is expressed in the body, tissue or in a cell upon administration of the compounds described herein as compared to the level or amount of expression of an appropriate control, e.g., the expression of a housekeeping mRNA and/or protein, e.g., GAPDH, and/or the expression of the biomarker prior to administration to a subject (or contacting a cell) of the compound of formula (A) or formula (I). In some embodiments, the expression is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In some embodiments, the expression of ANP is decreased by about 20%. In other embodiments, the expression of BNP is decreased by about 60%. In still other embodiments, the expression of MMP-2 is decreased by about 15%. In yet other embodiments, the expression of MMP-9 is decreased by about 50%. In still other embodiments, the expression of TIMP-1 is decreased by about 50%.

As used herein, the term “decreasing the activity”, e.g., of a biomarker of a myocardial disorder, refers to the reduction in the biological activity of the biomarker, e.g., cytokine production, ventricular function, myocardial remodeling, angiogenesis, in the body, tissue or in a cell upon a cell upon administration of the compounds described herein as compared to the activity of an appropriate control, e.g., the activity of the biomarker prior to administration to a subject (or contacting a cell) of the compounds described herein. In some embodiments, the activity is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%.

In some embodiments, the teachings provide methods for decreasing the expression and/or activity of TLR4 or CD14 or a molecule in a signal transduction pathway involving TLR4 and or CD14 in a subject suffering from a myocardial disorder by administering to the subject an effective amount of a compound of formula (A) or a compound of formula (I). In some embodiments, the teachings provide methods for decreasing the expression and/or activity of TLR4 or CD14 in a cell by contacting the cell with a compound of formula (A) or a compound of formula (I). As used herein, the term “TLR4” refers to toll-like receptor 4, a transmembrane protein of the toll-like receptor family, which plays a key role in pathogen recognition and activation of innate immunity. The term “CD14,” as used herein, refers to protein that exists as a glycosylphosphatidylinositol anchored membrane protein or as a soluble form and is a co-receptor with TLR4. Without wishing to be bound by any particular theory, it is believed that the upregulation of CD14 in a subject suffering from a myocardial disorder may be due to a release of fibrinogen. For example, increased fibrinogen can accumulate in the extracellular matrix of a hypertrophic heart induced by aortic banding (see, e.g., Li et al., J Hypertens 2009, 27: 1084-1093).

In some embodiments, the expression is decreased (as compared to an appropriate control) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In some embodiments, the expression of TLR4 is decreased by about 30%. In some other embodiments, the expression of CD14 is decreased by about 10%.

In some embodiments, the activity of a cytokine is decreased (as compared to an appropriate control) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%.

In some embodiments, the teachings provide methods for decreasing the expression and/or activity of a cytokine in a subject suffering from a myocardial disorder by administering to the subject an effective amount of a compound of formula (A) or a compound of formula (I). In some embodiments, the teachings provide methods for decreasing the expression and/or activity of a cytokine in a cell by contacting the cell with an effective amount of a compound described herein. Without wishing to be bound by any particular theory, it is believed that cytokines contribute to the development and progression of heart failure and play an important role in cardiac remodelling and dysfunction (see, e.g., Baumgarten et al., Trends Cardiovasc Med 2000, 10: 216-223). In some embodiments cytokines include, but are not limited to, interleukin (IL)-1β, IL-6 or IL-10. In one embodiment, provided herein are methods for decreasing the expression and/or activity of a pro-inflammatory cytokine (e.g., IL-1β or IL-6). In another embodiment, provided herein are methods for increasing the expression and/or activity of an anti-inflammatory cytokine (e.g., IL-10).

In some embodiments, the expression of a cytokine is decreased (as compared to an appropriate control) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In some embodiments, the expression of IL-6 is decreased by about 25%. In some other embodiments, the expression of IL-1β is decreased by about 75%.

In some embodiments, the activity is decreased (as compared to an appropriate control) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%.

The nucleotide and amino acid sequence of the genes and proteins of interest are known in the art and assays for determining the expression of a gene (e.g., RNA) or protein of interest, e.g., a biomarker, are well known in the art. Such assays include, but are not limited to, immunological methods for detection of proteins, protein purification methods, protein function or activity assays, nucleic acid hybridization methods, nucleic acid reverse transcription methods, and nucleic acid amplification methods, ELISA, immunoblotting, Western blotting, Northern blotting, electron microscopy, and Southern blotting.

Similarly, assays for determining the activity of a gene of interest are known in the art and include, but are not limited to, immunological methods for detection of proteins, protein purification methods, protein function or activity assays, nucleic acid hybridization methods, nucleic acid reverse transcription methods, and nucleic acid amplification methods, ELISA, immunoblotting, Western blotting, Northern blotting, electron microscopy, and Southern blotting, cell based assys of angiogenesis and mycocardial remodeling and animal models of ventricular function, myocardial remodeling, angiogenesis.

### Dosage Forms

It will be appreciated that the compounds described herein may be administered systemically in dosage forms, formulations or suitable delivery devices or implants containing conventional, non-toxic pharmaceutically acceptable carriers and adjuvants such that the compound effectiveness is optimized. For example, the compound of formula (A) or compound of formula (I) may be formulated together with appropriate excipients into a pharmaceutical composition, which, upon administration of the composition to the subject, systemically releases the active substance in a controlled manner. Alternatively, or additionally, compound dosage form design may be optimized so as to increase the effectiveness of the compound described herein upon administration. The above strategies (i.e., dosage form design and rate control of drug input), when used alone or in combination, can result in a significant increase in compound effectiveness and are considered part of the invention.

In some embodiments, the compound may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject.

Solid dosage forms for oral administration include capsules, tablets, pills, powders, and granules. In such solid dosage forms, the compounds described herein may mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The compounds described herein may also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions, which can be used, include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions, which are compatible with body tissues.

In still other embodiments, the compound of formula (A) or the compound of formula (I) is formulated in a manner that increases efficacy. For example, in some embodiments, the compound of formula (A) (e.g., the compound of formula (I)) is formulated to delay or decrease binding to high density lipoproteins. In other embodiments, the compound described herein is formulated to increase or enhance binding to low density lipoprotein.

It will also be appreciated that the compound of formula (A) or compound of formula (I) may be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound described herein may be administered concurrently with another agent that treats or prevents a myocardial disorder), or they may achieve different effects (e.g., control of any adverse effects).

### Exemplification of the Invention

The methods of this invention can be understood further by the following examples. It will be appreciated, however, that these examples do not limit the invention. Variations of the invention, now known or further developed, are considered to fall within the scope of the present invention as described herein and as hereinafter claimed.

### EXAMPLE 1

### Methods

11 to 13 week old male C57BL/6 mice were purchased from Charles River (Germany). Mice were housed in individually ventilated pathogen-free cages with free access to water and standard rodent chow. The animals were handled according to the principles of laboratory animal care (NIH publication No. 85-23, revised 1996), and animal procedures were approved by the local committee for animal care.

To allow for repetitive intravenous injections, a venous port was implanted. Mice were anesthetized with isoflurane 2.0 vol % (Forene®, Abbott GmbH, Germany). Body temperature was maintained at 37° C. The jugular vein was exposed and a catheter (outer diameter 0.64 mm, Dow Corning Europe, Wiesbaden, Germany) inserted into the vessel via a small incision. The catheter was secured with a silk thread and tissue adhesive (Histoacryl, Braun, Germany). Finally, the catheter was tunneled subcutaneously and connected to an extracutaneous port through an incision between scapulae.

The first injection was performed 5 min prior to TAC or sham-operation procedure (described below) and repeated 4, 8, 12, 24, 36, 48 and 60 hrs after surgery. Animals were randomly assigned into two groups, receiving either a tetrasodium salt of the compound of formula 1 (hereinafter “Eritoran tetrasodium”) or placebo. The catheter was flushed with 50 μl 0.9% NaCl before and 100 μl after each injection of 50 μl Eritoran tetrasodium (2.5 mg/ml vehicle; 5 mg/kg bodyweight/application; dosage adopted from Shimamoto et al., Circulation 2006, 114: 1270-1274) or placebo solution.

Treatment groups were separated into two subgroups, undergoing transverse aortic constriction (TAC) or sham operation. Surgery for TAC was achieved as published previously (see, e.g., Baumgarten et al., Circulation 2002, 105: 2192-2197 and Baumgarten et al., Basic Res Cardiol 2006, 101: 427-435). Mice were intubated in a supine position and mechanical ventilation was initiated (MiniVent 845, Hugo Sachs Elektronik, Germany). A left parasternal incision was performed. A suture was passed underneath the aorta and tied down on a 27 Gauge needle, which was immediately removed and allowed to achieve a standardised and previously validated decreased diameter of the aorta. For sham operation the suture was passed underneath the aorta without ligation. For analgesia, mice received a single intraperitoneal injection of 0.065 mg/kg BW buprenorphine.

Three days after TAC or sham operation, the impact on cardiac biometric parameters was investigated. Body weight was registered. Heart and lung were excised, prepared and immediately measured. Total heart weight, left ventricular weight as well as lung weight and tibia length were recorded. Ventricles were snap frozen in liquid nitrogen and kept at −80° C.

Quantitative real-time PCR analysis was completed. For total RNA extraction, left ventricles were homogenized and RNA was isolated using the thiocyanate-phenol-chloroform method (see, e.g., Chomczynski et al., Anal Biochem 1987, 162: 156-159). RNA was dissolved in 100 μl of RNase-free water, and concentration was determined photometrically (absorbance at 260 nm) before storage at −80° C. RNA was reverse transcribed using High Capacity cDNA Reverse Transcription Kit (Applied Biosystems, CA, USA, Part No. 4368814) according to the manufacturer's protocol. 25 μl RNA were mixed with 25 μl master mix, containing 5 μl 10× reverse transcriptase buffer, 2 μl 25× dNTPs, 2 μl 10× random primers, 2.5 μl multi scribe reverse transcriptase and 10.5 μl nuclease free water.

The specific pre-made TaqMan® Gene Expression Assays (Applied Biosystems) used are listed in Table 1. Real-time PCR was performed according to the manufacturer's protocol. 5.5 ng of cDNA was mixed with 5 μl 2× TaqMan® Universal Master Mix (Applied Biosystems, #4304437), 0.5 μl TaqMan® Gene Expression Assay and 2.3 μl nuclease free water to a final volume of 10 μl in a 384-well optical reaction plate. Each sample was measured in triplicate wells and underwent 40 cycles of amplification on an ABI PRISM® Sequence Detection System (Applied Biosystems). C_{T} values were determined with SDS Software 2.2 (Applied Biosystems) and relative quotients (RQ) were calculated following the ΔΔC_{T} method (RQ target gene/GAPDH).

TABLE 1
TaqMan ® gene expression assays used for real-time PCR.

| | |
|---|---|
| ANF | Mm01255748_g1 |
| BNP | Mm01255770_g1 |
| CD14 | Mm00438094_g1 |
| GAPDH | Mm99999915_g1 |
| IL-1β | Mm99999061_mH |
| IL-6 | Mm01210732_g1 |
| IL-10 | Mm00439616_m1 |
| MMP-2 | Mm00439508_m1 |
| MMP-9 | Mm00442991_m1 |
| TIMP-1 | Mm00441818_m1 |
| TIMP-4 | Mm00446568_m1 |
| TLR-2 | Mm01213946_g1 |
| TLR-4 | Mm00445273_m1 |
| TNF-α | Mm00443258_m1 |
| TTP | Mm00457144_m1 |

For ELISA assays, proteins from snap-frozen myocardial tissue were isolated and cytosolic and nuclear proteins separated according to the manufacturer's protocol (NE-PER®, Nuclear and Cytoplasmic Extraction Kit, Pierce®, Thermo Scientific, IL, USA). Myocardial protein levels in the cytoplasmic fraction were detected with Quantikine mouse IL-1β as well as IL-6 ELISA (R&D Systems, McKinley, USA) as described in the manufacturer's protocol. Cytokine concentration was normalized versus protein concentration in the supernatant. Protein concentration was determined by B CA protein assay (Pierce®).

For zymographic measurement of MMP activity, protein isolates from left ventricular tissue (60 μg protein content, isolated with NE-PER protein isolation kit; Pierce®) were mixed with 2× Tris-Glycine SDS sample buffer, and loaded on precast zymogram gels (SDS-PAGE; BioRad, Germany) containing 10% gelatine. Following 120 min of electrophoresis at 125 V, gels were washed with 2.5% Triton X-100 (Sigma Aldrich, Germany) for 3×20 min to remove SDS. Then, gels were incubated for 48 h at 37° C. in developing buffer (50 mM Tris-HCl, 0.2 M NaCl, 5 mM CaCl₂, 0.02% Brij). Gels were stained using 0.1% (w/v) Brilliant Blue (Sigma) in a mixture of water:methanol:acetic acid (5:5:1 v/v), destained in 45% methanol, and 3% acetic acid in water (v/v). Areas of protease activity were detected as transparent bands against blue background.

Statistics were calculated using Prism 4.05 (GraphPad Software Inc., CA, USA). All values are expressed as mean (M)±SEM. One-way ANOVA with Newman-Keuls post-hoc testing was performed for statistical analysis with the exception of TIMP and cytokine expression which were analysed with Kruskal-Wallis and Dunn's multiple comparison post-hoc testing Impact of TAC-Eritoran on IL-10 expression was analysed with Two-way ANOVA. Differences between experimental groups were considered to be significant with p<0.05.

### Results

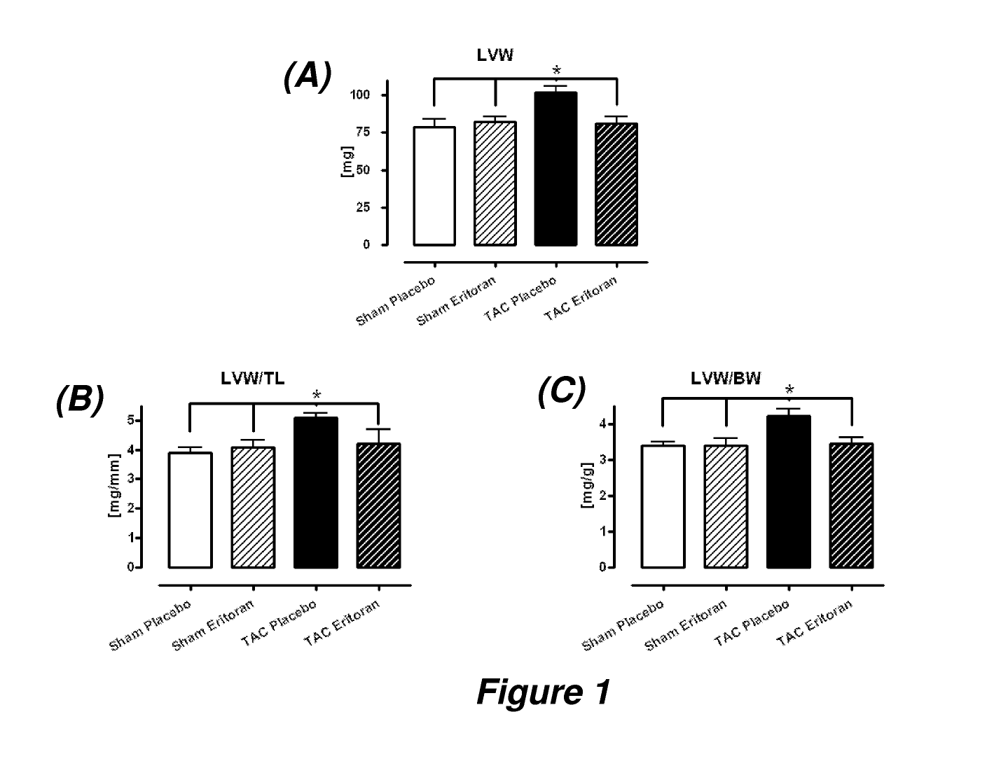

It was found that Eritoran tetrasodium decreases cardiac hypertrophy after TAC. Three days after TAC surgery a significant increase of left ventricular weight was detected (FIG. 1A). Interestingly, in the Eritoran tetrasodium-treated group heart weight was significantly lower (LVW 81.32±4.21 mg) versus TAC placebo (LVW 101.7±4.53 mg), p<0.05. Mice were age and weight matched. Thus, normalization of LVW to body weight (BW) or to tibia length (TL) confirmed that aortic banding accounted for LVW differences between TAC and placebo groups (FIGS. 1B and 1C). Pressure overload induced significantly increased LVW/BW and LVW/TL in mice without Eritoran tetrasodium treatment (LVW/BW: 3.45±0.16 mg/g vs. 4.21±0.21 mg/g, p<0.05; LVW/TL: 4.21±0.19 mg/mm vs. 5.08±0.16 mg/mm, p<0.05), while biometric results obtained from Eritoran tetrasodium treated TAC mice did not differ from sham-operated mice.

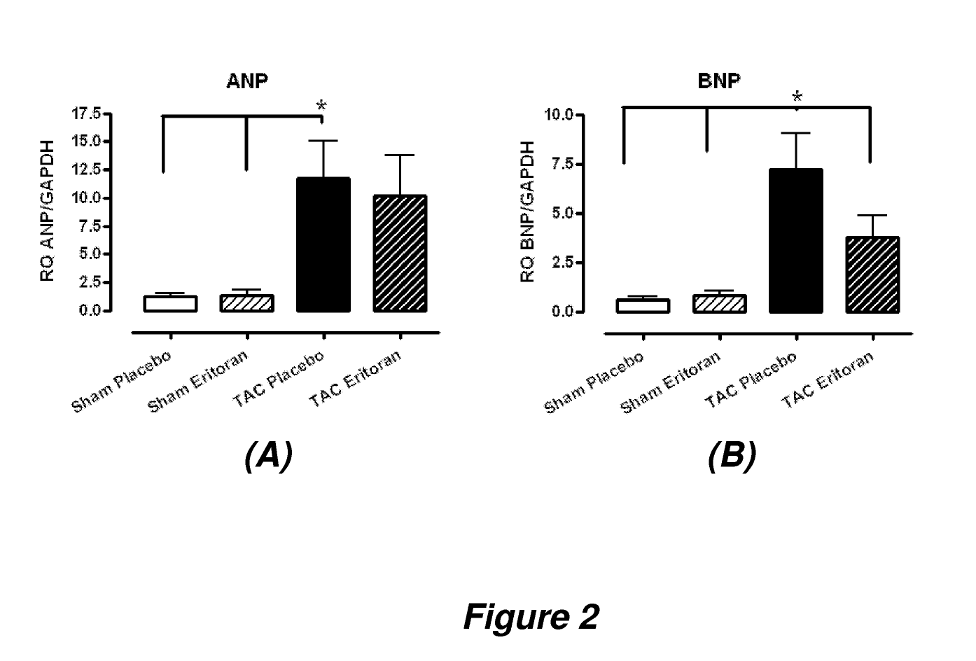

Regarding mRNA expression of natriuretic peptides ANP and BNP, samples analysed with molecular biological methods were essentially identical to those used for biometric measurements (above). Pressure overload induced an upregulation of the hypertrophy markers ANP and BNP (FIG. 2), while sham-operated mice expressed little ANP or BNP. Within placebo groups, TAC increased both ANP and BNP levels more than tenfold (ANP: p<0.05, BNP: p<0.01). After aortic banding, Eritoran tetrasodium markedly decreased BNP but not ANP expression (BNP:TAC placebo: 7.73±2.18 vs. TAC Eritoran tetrasodium: 3.73±1.15, p<0.05).

It was also found that TAC induced matrix metalloproteinase activity was attenuated following Eritoran tetrasodium application. MMPs and their specific inhibitors regulate extracellular matrix degradation and synthesis, thereby controlling cardiac remodeling.

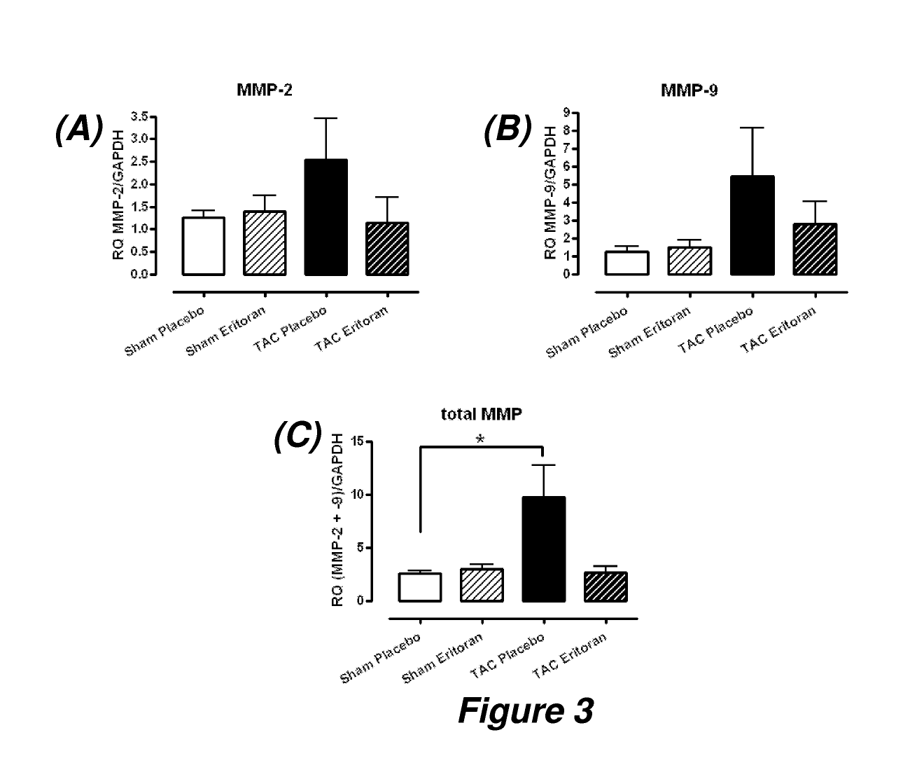

Regarding mRNA expression of MMP-2, and MMP-9, qRT-PCR revealed an overall increase of both MMP-2 and MMP-9 expression levels after TAC in mice without Eritoran tetrasodium treatment (FIG. 3). Total MMP mRNA expression of MMP-2 and MMP-9 were calculated, and revealed a significant elevation of total MMP mRNA after TAC in the placebo group (sham placebo: 2.52±0.28 vs. TAC placebo: 9.68±3.05, p<0.05). Single and total MMP mRNA levels of Eritoran tetrasodium-treated groups remained unchanged by aortic banding.

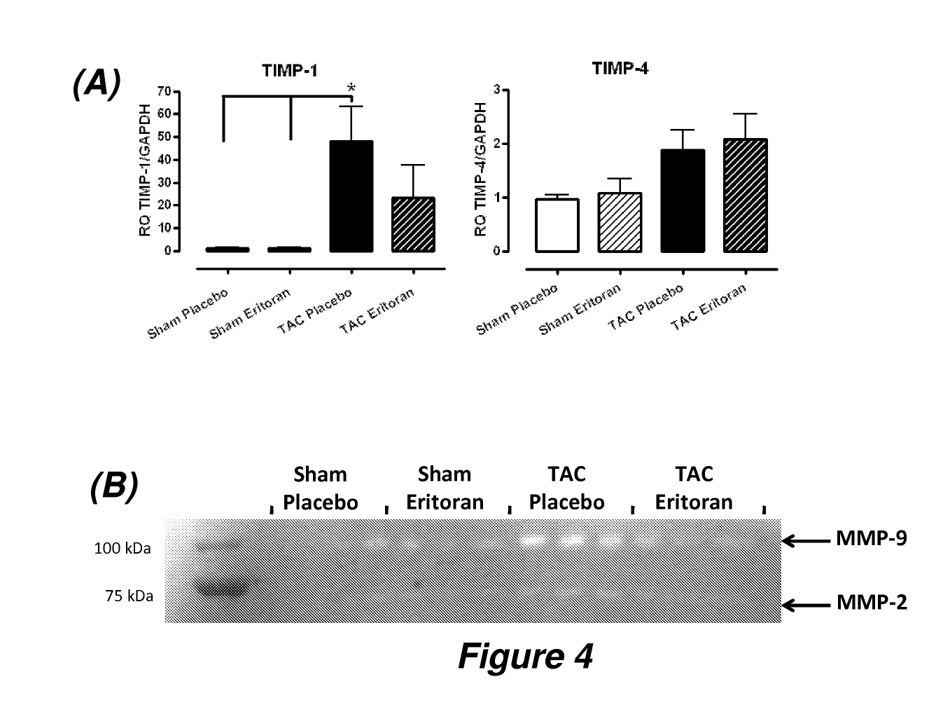

Regarding mRNA expression of TIMP-1 and TIMP-4, FIG. 4(A) illustrates that TIMP-1 mRNA expression increased after TAC and reached the level of significance in the placebo group (sham placebo: 1.12±0.37 vs. TAC placebo: 48.19±15.14, p<0.05; sham Eritoran tetrasodium: 1.16±0.40 vs. TAC Eritoran tetrasodium: 23.20±14.52, not significant). TIMP-4 induction was only mild and showed no significant differences between groups.

Regarding zymographic activity of MMP-2 and MMP-9, FIG. 4(B) depicts MMP-2 and MMP-9 activities detected with gelatine zymography. In accordance with the mRNA expression levels, MMP-2 activity was less pronounced than MMP-9 activity. Gelatine degradation by MMP-2 after TAC was only modestly increased. In contrast, a distinct induction of MMP-9 zymographic activity was observed in TAC-placebo samples.

It was also found that Eritoran tetrasodium modulates the pro- and anti-inflammatory responses after TAC. It has been shown, that sustained pressure overload provokes a transient increase in proinflammatory cytokine expression (see, e.g., Baumgarten et al. Circulation 2002, 105: 2192-2197). Therefore, gene expression of pro-(TNFα, IL-1β, IL-6) and anti-inflammatory cytokines, as well as TTP, were analyzed (Table 2).

Eritoran tetrasodium had no effect on the basal expression of proinflammatory cytokines. Three days after TAC, TNF-α mRNA levels increased slightly compared to sham-operated groups (sham placebo: 0.73±0.11 vs. TAC placebo: 1.13±0.18; sham Eritoran tetrasodium: 0.84±0.22 vs. TAC Eritoran tetrasodium: 1.32±0.19, not significant).

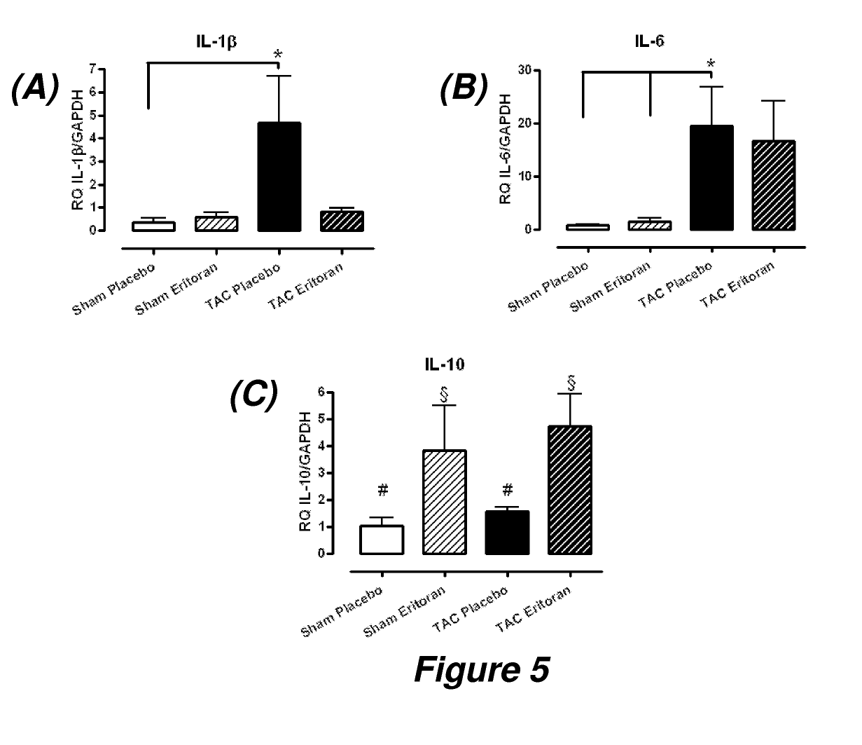

IL-1β and IL-6 mRNA expression were elevated by aortic banding. Compared to sham-operated placebo mice, IL-1β and IL-6 expression were increased 13- and 28-fold, respectively (p<0.05). TLR4 antagonism completely inhibited TAC induced IL-1β mRNA expression, which was similar to sham-operated mice (FIG. 5). Interestingly, Eritoran tetrasodium caused a three to four fold elevation of the anti-inflammatory cytokine IL-10 in sham and TAC groups (p<0.05, Two-way Anova). Pressure overload alone had no significant effect on IL-10 expression.

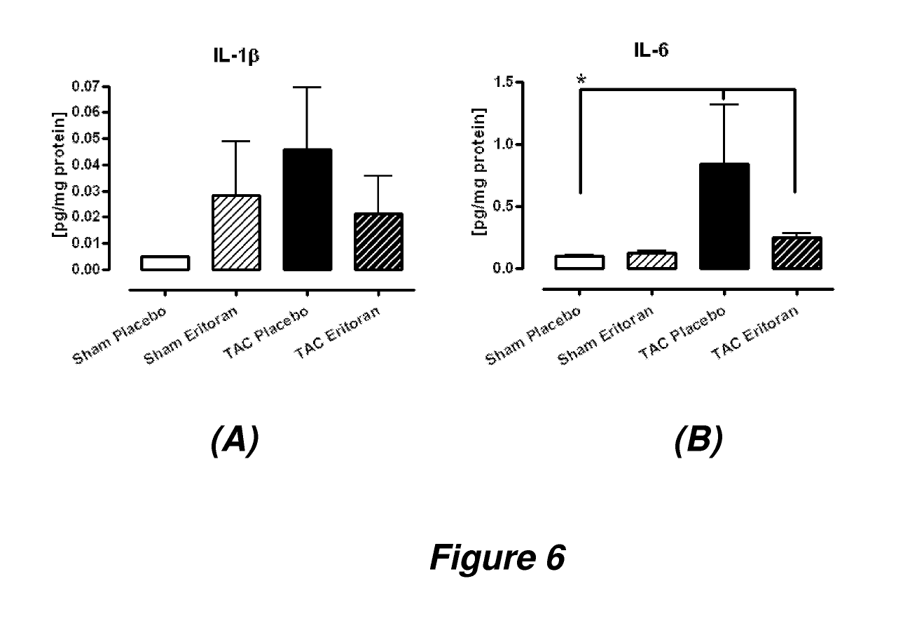

IL-1β and IL-6 protein expression were analysed with ELISA. Within placebo groups, protein induction after TAC followed the observed elevation of mRNA data. The amount of IL-6 protein in TAC groups differed significantly from the placebo sham group. However, TAC Eritoran tetrasodium did not display significant increases of IL-1β or IL-6 compared to the respective Eritoran tetrasodium treated sham group. Overall, TAC Eritoran tetrasodium samples exhibited lower levels of pro-inflammatory cytokines compared to TAC placebo hearts (not significant) (FIG. 6).

Tristetraprolin (TTP) binds to AU-rich elements in the 3′ untranslated region of various cytokine mRNAs and destabilizes it (see, e.g., Carballo et al., Science 1998, 281: 1001-1005). Pressure overload regulates TTP and thereby potentially decreases TNFα mRNA levels (see, e.g., Baumgarten et al. Circulation 2002, 105: 2192-2197 and Hikoso et al., Circulation 2004, 110: 2631-2637). Therefore, these parameters were also analysed in this model. Three days after TAC surgery, elevation of TTP mRNA was not significant (Table 2). A slight increase was found in both Eritoran tetrasodium treated groups. However, the TTP/TNFα ratio did not reveal a potentially beneficial shift towards TTP in the Eritoran tetrasodium TAC group. TLR4 antagonism only induced a modest increase of the TTP/TNFα value in the sham group (sham placebo: 1.03±0.29, sham Eritoran tetrasodium: 1.40±0.33, TAC placebo: 0.82±0.20, TAC Eritoran tetrasodium: 0.61±0.33; not significant, data not shown). However, it is believed that TTP mRNA expression may be transiently induced early (e.g., a few hours) after TAC, thus elevating TTP protein after 3 days.

TABLE 2
Effect of hemodynamic overloading pro- and anti-inflammatory
cytokine, and TTP mRNA levels determined by qRT-PCR
(M ± SEM; n = 5-7). Different characters (a, b) indicate
significant differences between the labelled groups (p < 0.05).

| | | Sham Eritoran | | TAC Eritoran |
|---|---|---|---|---|
| | Sham Placebo | tetrasodium | TAC Placebo | tetrasodium |
| TNF | 0.73 ± 0.11 | 0.84 ± 0.22 | 1.13 ± 0.18 | 1.32 ± 0.19 |
| IL-1β | 0.36 ± 0.17 a | 0.60 ± 0.17 | 4.69 ± 2.00 b | 0.81 ± 0.17 |
| IL-6 | 0.70 ± 0.26 a | 1.44 ± 0.65 a | 19.44 ± 7.26 b | 16.60 ± 7.55 |
| IL-10 | 1.03 ± 0.28 a | 3.81 ± 1.69 b | 1.55 ± 0.18 a | 4.71 ± 1.22 b |
| TTP | 0.84 ± 0.28 | 1.08 ± 0.39 | 0.80 ± 0.17 | 0.96 ± 0.52 |

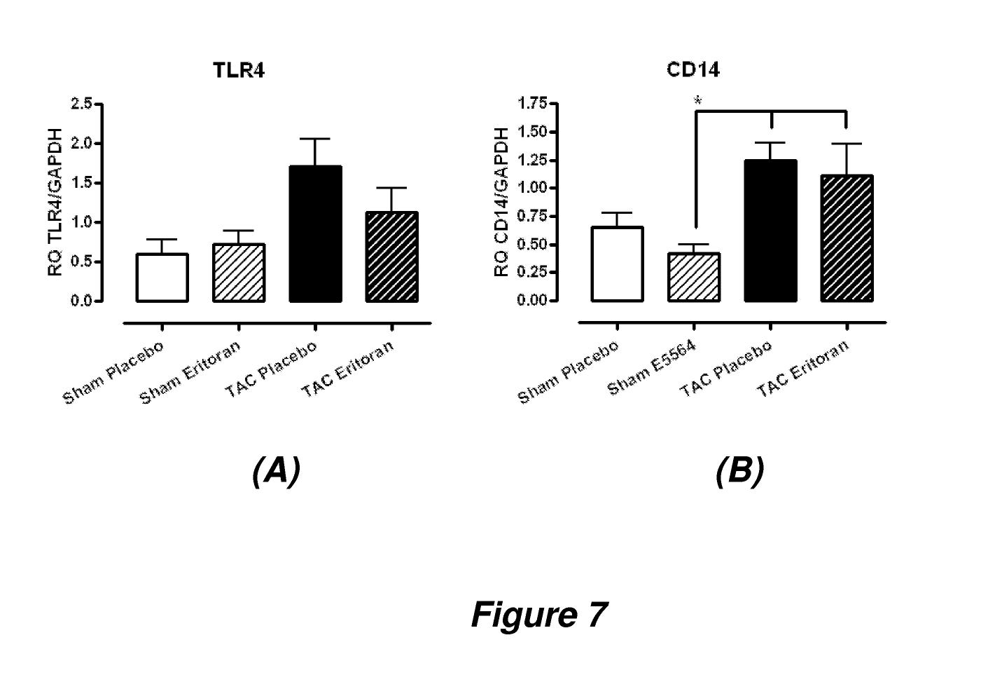

It has been shown that three days of cardiac pressure overload modulate the expression of the TLR4/CD14 complex (Baumgarten et al., Basic Res Cardiol 2006, 101: 427-435). Therefore, mRNA expression of TLR4 and CD14 (FIG. 7) was analyzed. In the sham group, Eritoran tetrasodium had no effects on the mRNA expression. TAC induced an increase of TLR4 (sham placebo: 0.6±0.18 vs. TAC placebo: 1.71±0.35, not significant) and CD14 mRNA (sham placebo: 0.65±0.13 vs. TAC placebo: 1.24±0.16, not significant). However, a significant difference of CD14 mRNA expression induced by TAC was found between sham Eritoran tetrasodium and the respective TAC group (sham Eritoran tetrasodium: 0.42±0.08 vs. TAC Eritoran tetrasodium: 1.11±0.28). Eritoran had no significant effects on TLR4 or CD14 expression after TAC. Other endogenous ligands released due to organ injury are potentially recognized by TLR2 and might regulate receptor expression. However, in this study cardiac pressure overload did not change TLR2 expression. All values remained within the range of baseline expression, independent of any treatment (sham placebo: 0.87±0.06 vs. TAC placebo: 0.88±0.24, data not shown).

### Conclusion

Eritoran tetrasodium treatment diminished cardiac hypertrophy induced by pressure overload, attenuated the increase of the natriuretic peptide BNP, averted IL-1β and IL-6 pro-inflammatory cytokine expression and prevented remodeling mechanisms due to reduced zymographic activity of MMP-9.

### EXAMPLE 2

Example 2 was completed in a manner similar to Example 1, with minor changes as described below.

### Methods

With the consent of proper authority (LANUV), 12 week old male C57BL/6-mice were anesthetized with Isofluran (2.0 Vol. %) and a catheter was implanted into the right jugular vein. Afterwards, either transverse aortic constriction (TAC) or a sham operation was performed. In TAC animals, the aortic diameter was reduced by 70% with a 6-0 silk filament. Mice received either the compound of formula (I) or placebo i.v. 5 minutes prior to TAC/sham-operation and 6 h, 12 h, 24 h, 36 h, 48 h and 60 h after surgery. Three days after surgery hearts were taken, the left ventricle weight (LVW) was determined and cardiac tissue was prepared for mRNA and protein analysis via qRT-PCR, ELISA, and Western Blot and for zymographic assay (statistics: one-way ANOVA and Bonferroni post-hoc analysis; p≦0.05 was considered significant; n=6/group).

### Results

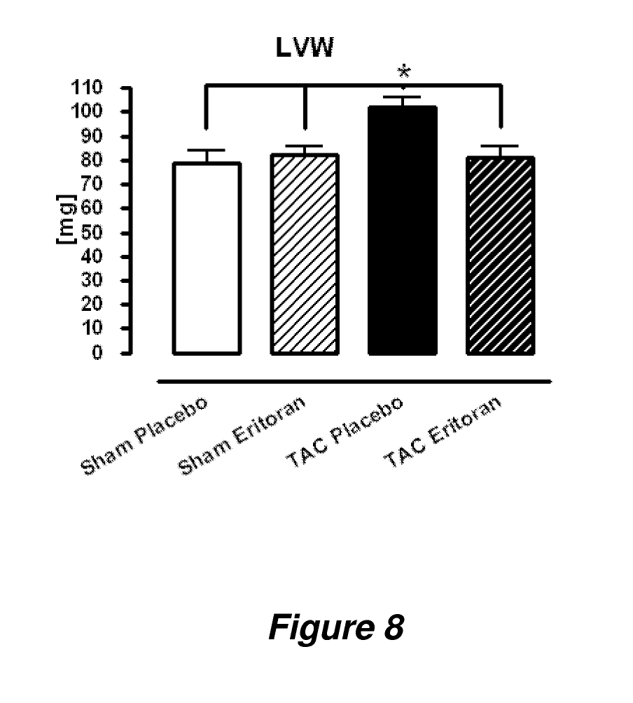

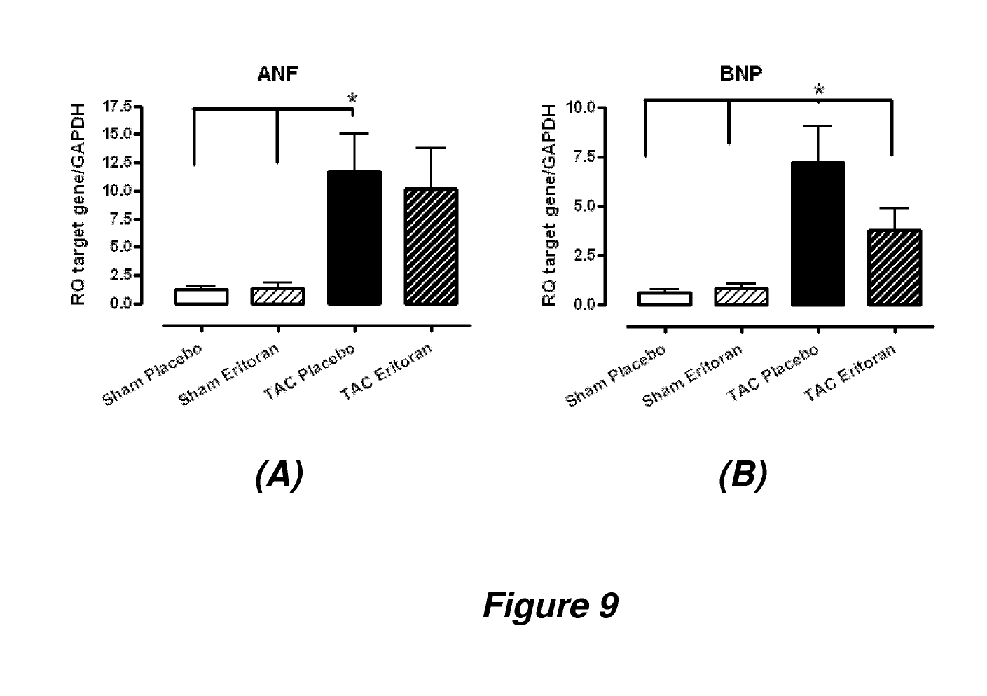

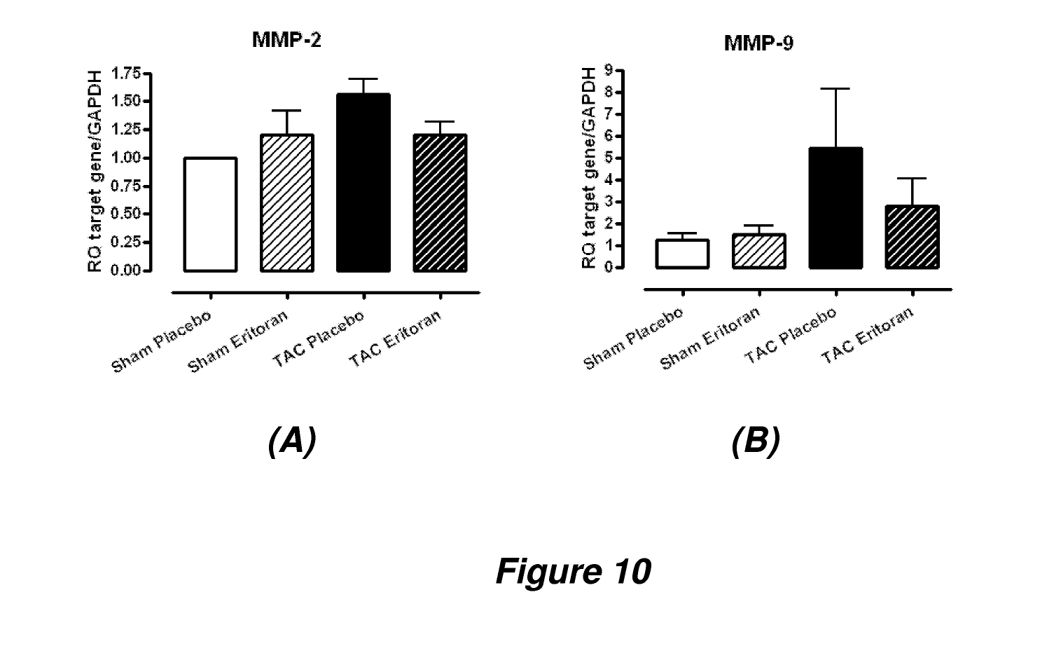

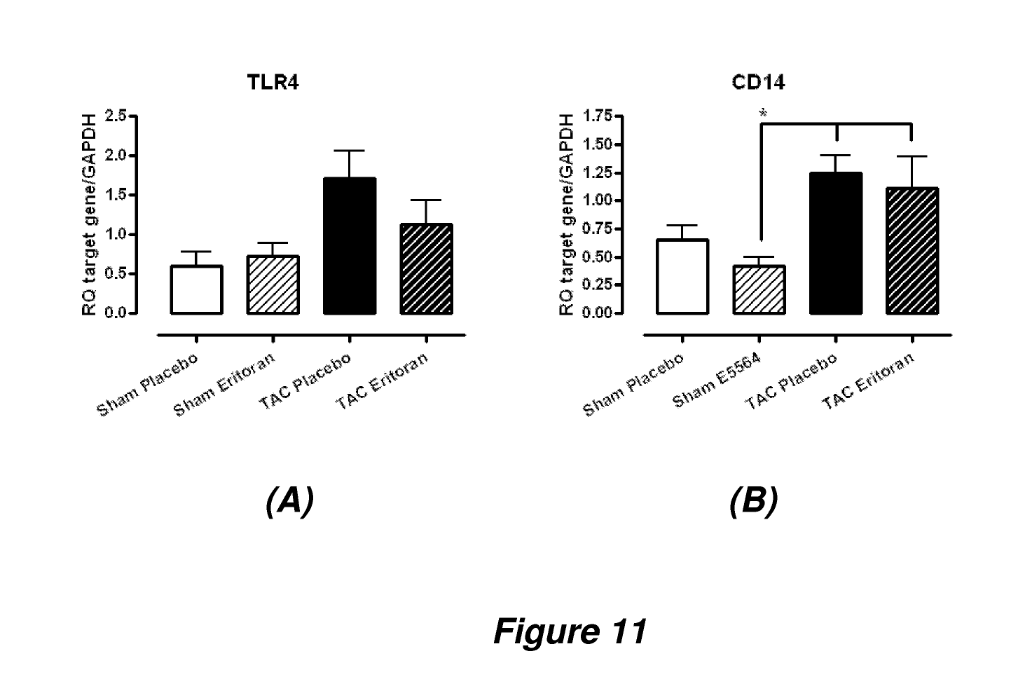

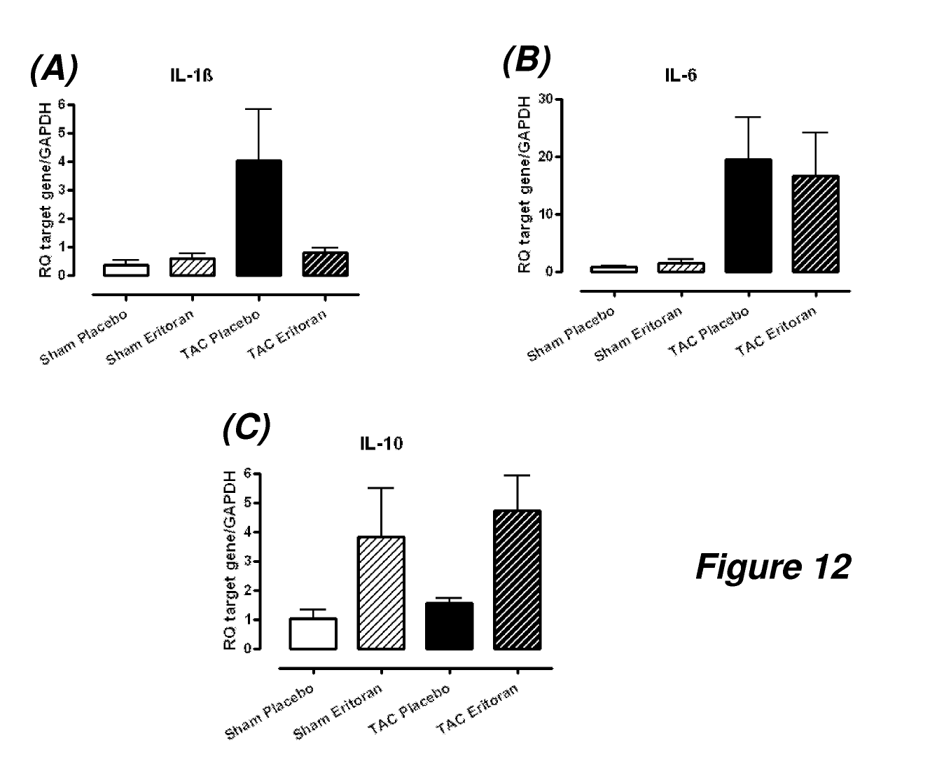

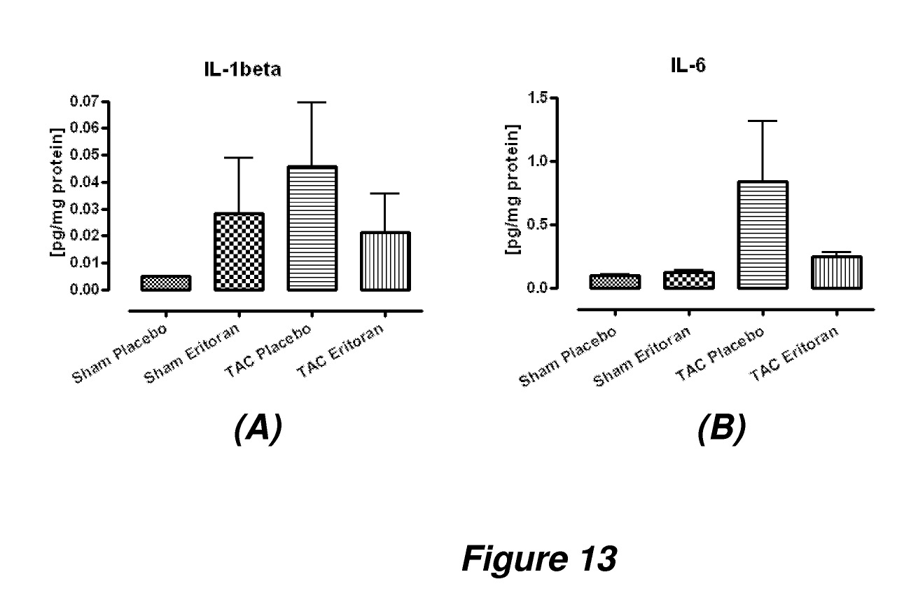

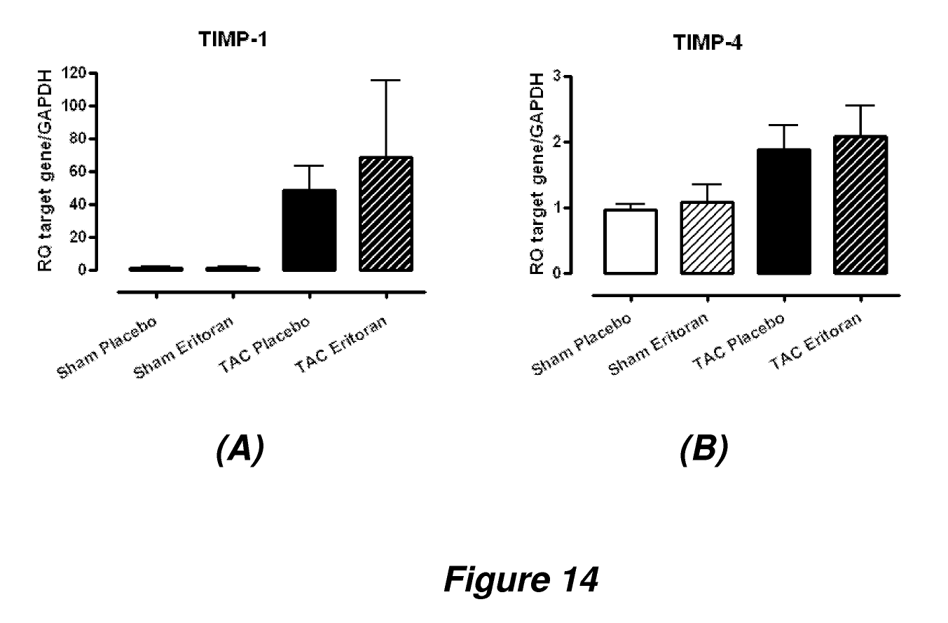

As shown in FIG. 8, the TAC-placebo group displayed an increase of LVW of 33% (LVW; 104.2±5.5 mg) which was significantly higher compared to sham (78.74±1.9 bzw. 82.45±3.5 mg). In contrast, there was no such increase of LVW in the TAC group treated with compound I (81.32±4.2 mg). Additionally, the quotient of LVW/tibia length was significantly different from all other TAC-groups. The mRNA-expression of atrial natiuretic peptide (ANP) and brain natriuretic peptide (BNP), markers of cardiac hypertrophy, were elevated in TAC-placebo mice compared to sham and the TAC groups treated with the compound of formula (I) (see FIGS. 9(A) and (B), respectively). The mRNA expression of matrix metalloproteinase-2 (MMP-2) and -9 (MMP-9) activity was higher in the TAC-placebo animals compared to the TAC groups treated with the compound of formula (I) (see FIGS. 10(A) and (B), respectively), as was the MMP-9 zymographic activity. As shown in FIG. 11(A), mRNA expression of TLR4 was also significantly reduced in the TAC groups treated with the compound of formula (I) compared to the TAC-placebo group, although the decrease in mRNA expression of CD14 (FIG. 11(B)) was not as great. FIGS. 12(A) and (B) indicate that mRNA expression IL-6 and IL-1β was lower in the TAC groups treated with the compounds of formula (I) when compared with the TAC-placebo group, while the mRNA expression of IL-10 increased (FIG. 12(C)). The protein expression of IL-6 and IL-1β was also reduced in the TAC group treated with the compound of formula (I) (see FIGS. 13(A) and (B)). Finally, mRNA expression of the tissue inhibitors of matrix metalloproteinases-1 (TIMP-1) and -4 (TIMP-4) in the TAC groups treated with the compound of formula (I) was higher when compared to the mRNA expression in the TAC-placebo group (see FIGS. 14(A) and (B), respectively).

### Conclusion

Administration of the TLR4-antagonist Eritoran prevents the development of cardiac hypertrophy in a murine model of transverse aortic constriction (TAC).

### Equivalents

Those skilled in the art will recognize, or be able to ascertain using no more than routine experimentation, many equivalents to the specific embodiments of the invention described herein. Such equivalents are intended to be encompassed by the following claims.

1. A method for treating a myocardial disorder in a subject comprising administering to said subject an effective amount of a compound of formula (I): 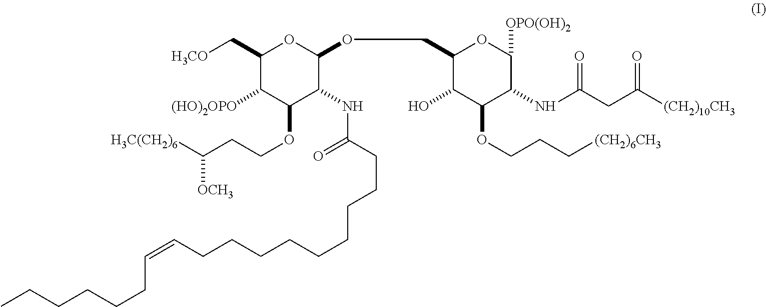
or a pharmaceutically acceptable salt thereof, such that said myocardial disorder is treated.

2. A method for treating a subject at risk of suffering from a myocardial disorder comprising administering to said subject an effective amount of a compound of formula (I): 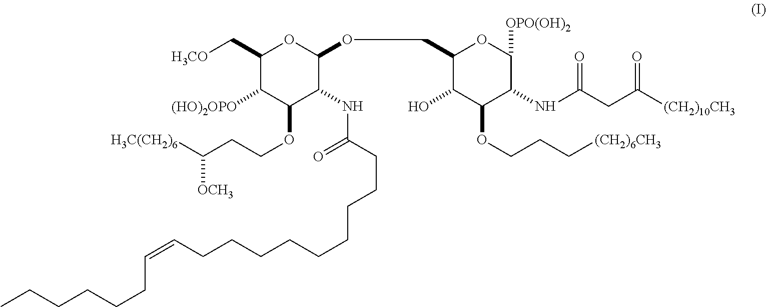
or a pharmaceutically acceptable salt thereof, such that said risk is reduced.

3. A method for preventing a myocardial disorder in a subject comprising administering to said subject an effective amount of a compound of formula (I): 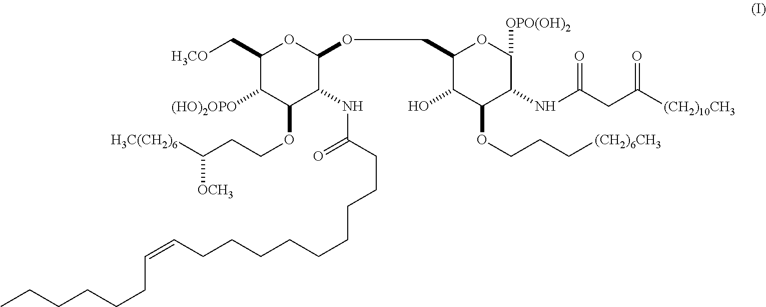
or a pharmaceutically acceptable salt thereof, such that said myocardial disorder is prevented.

4. The method of any one of claims 1-3, wherein said myocardial disorder is cardiac hypertrophy or a myocardial infarction.

5. The method of claim 4, wherein said cardiac hypertrophy is left ventricle hypertrophy or right ventricle hypertrophy.

6. The method of any one of claims 1-3, wherein said subject suffers from hypertension, aortic stenosis, hypertrophic cardiomyopathy, emphysema, cystic fibrosis, chronic bronchitis, sleep apnea, chronic pulmonary embolism, heart failure, irregular heart rhythm, angina or cocaine addiction.

7. A method for preventing an increase in myocardial mass in a subject suffering from cardiac hypertrophy comprising administering to said subject an effective amount of a compound of formula (I): 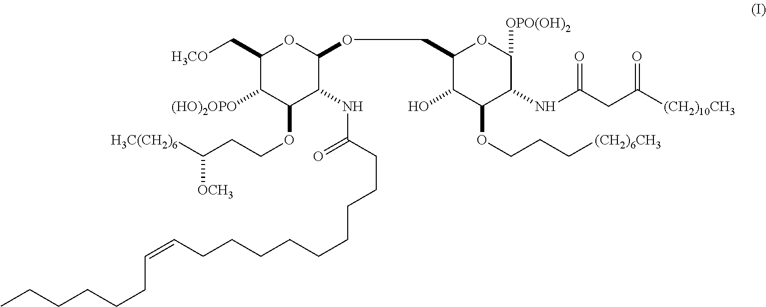
or a pharmaceutically acceptable salt thereof, such that said increase in myocardial mass is prevented.

8. A method for decreasing myocardial mass in a subject suffering from cardiac hypertrophy comprising administering to said subject an effective amount of a compound of formula (I): 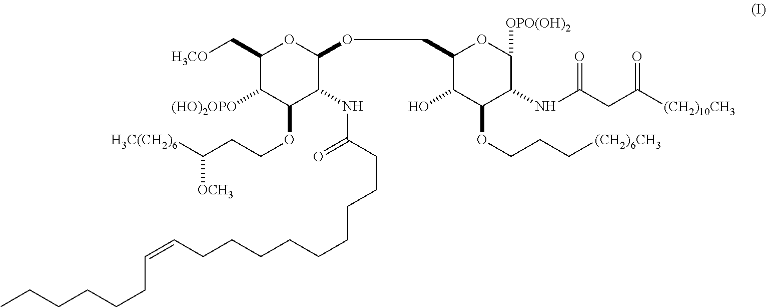
or a pharmaceutically acceptable salt thereof, such that myocardial mass is decreased.

9. A method for decreasing expression or activity of a biomarker of a myocardial disorder in a cell or in a subject suffering from a myocardial disorder comprising:
contacting the cell with or administering to the subject an effective amount of a compound of formula (I): 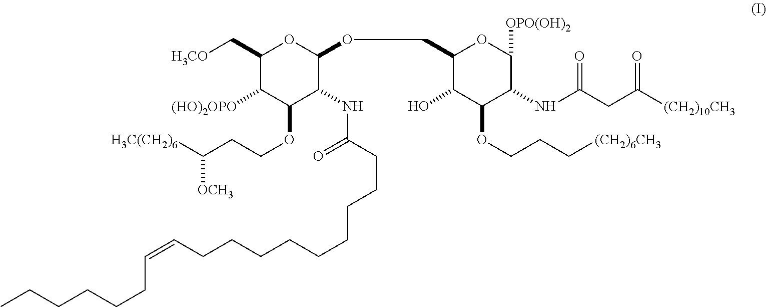
or a pharmaceutically acceptable salt thereof, such that said expression or activity is decreased.

10. The method of claim 9, wherein said biomarker is selected from the group consisting of atrial natriuretic peptide, brain natriuretic peptide, matrix metalloproteinase-2, matrix metalloproteinase-9 and tissue inhibitor of metalloproteinase-1.

11. A method for decreasing expression or activity of TLR4 or CD14 in a cell or in a subject suffering from a myocardial disorder comprising:
contacting the cell with or administering to the subject an effective amount of a compound of formula (I): 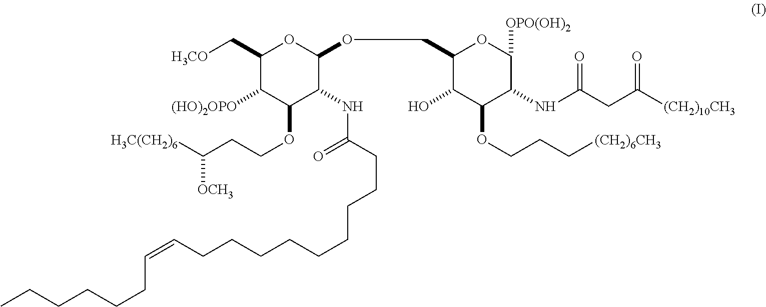
or a pharmaceutically acceptable salt thereof, such that said expression or activity is decreased.

12. A method for decreasing expression or activity of a cytokine in a cell or in a subject suffering from a myocardial disorder comprising:
contacting the cell with or administering to the subject an effective amount of a compound of formula (I): 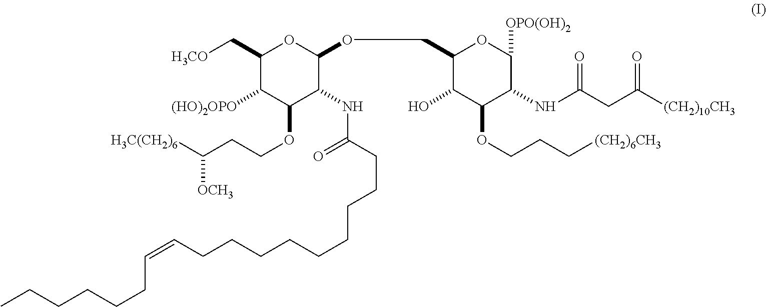
or a pharmaceutically acceptable salt thereof, such that said expression or activity is decreased.

13. The method of claim 12, wherein said cytokine is interleukin (IL)-1β, IL-6 or IL-10.

14. A method for treating left ventricle cardiac hypertrophy in a subject comprising administering to said subject an effective amount of a compound of formula (I): 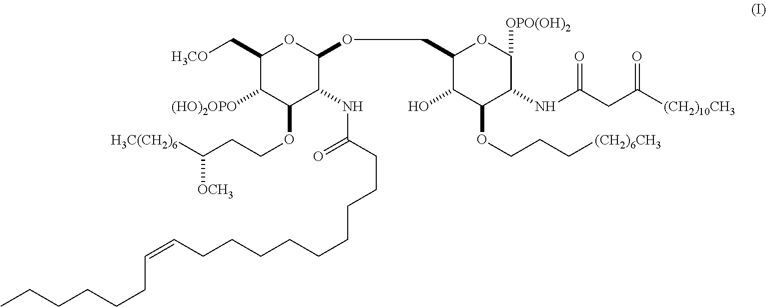
or a pharmaceutically acceptable salt thereof, such that said cardiac hypertrophy is treated.

15. The method of any one of the preceding claims, wherein the compound of formula (I) is a sodium salt.

16. The method of any one of the preceding claims, wherein the compound of formula (I) is administered systemically in a twice or thrice daily dose, or in an intermittent infusion.
